(19) United States
(12) Patent Application Publication
(10) Pub. No.: US 2025/0066727 A1
(43) Pub. Date: Feb. 27, 2025

(54) COMPOSITIONS AND METHODS FOR CULTURING STEM CELLS

(71) Applicant: Beijing TheraXyte BioScience Co. Ltd., Beijing (CN)

(72) Inventor: Tong ZHAO, Beijing (CN)

(21) Appl. No.: 18/725,324

(22) PCT Filed: Dec. 29, 2022
(86) PCT No.: PCT/CN2022/143169

§ 371 Date: Jun. 28, 2024

(30) Foreign Application Priority Data
Dec. 31, 2021 (WO) PCT/CN2021/143479

(51) Int. Cl. C12N 5/071

(52) U.S. Cl. CPC C12N 5/063; C12N 2500/32; C12N 2500/90; C12N 2501/11; C12N 2501/117; C12N 2501/15; C12N 2533/52; C12N 2533/54; C12N 2533/90

(57) ABSTRACT

Disclosed here is a serum-free composition, comprising: a coating matrix, a basal medium, and a growth supplement, as well as methods for making and using the serum-free composition. Also disclosed are kits comprising a coating matrix, a basal medium, and a concentrated stock of a growth supplement.

### CROSS-REFERENCE TO RELATED APPLICATIONS

This application claims priority to PCT/CN2021/143479, filed Dec. 31, 2021, which is incorporated herein by reference in its entirety.

### TECHNICAL FIELD

The disclosure relates to compositions and methods for culturing stem cells.

### BACKGROUND

The skin is an indispensable barrier that safeguards the body from the external environment. It possesses the ability to self-renew, which enables the replacement of dead cells and the repair of wounds, thereby sustaining the barrier function. It is made up of three layers, the epidermis, the dermis, and the hypodermis, all three of which vary significantly in their anatomy and function (FIG. 1). The layers of the epidermis include stratum basale, stratum spinosum, stratum granulosum, stratum lucidum, and stratum corneum. The dermis is connected to the epidermis at the level of the basement membrane, houses the sweat glands, hair, hair follicles, muscles, sensory neurons, and blood vessels. The hypodermis is deep to the dermis and is also called subcutaneous fascia.

Keratinocytes are the predominant cell type of epidermis and originate from keratinocyte stem cells (KSCs) located in the basal layer. KSCs can self-renew and differentiate to other types of keratinocytes, which are responsible for maintaining the epidermal homeostasis and for repairing the tissue following injuries. In normal circumstances, most cutaneous wounds heal without medical interventions. However, if the wound is extensive and extends into the dermis, medical attention may be required. Traditionally, the therapeutic strategy for treating large, deep wounds has been to use split-thickness skin autografts. However, this treatment is not viable in the case of extensive burn injury, as patients may lack sufficient healthy donor sites. Therefore, the grafting of cultured keratinocytes can be an alternative treatment to assist in the repair of damaged skin.

Keratinocytes can be expanded in vitro from a patient's skin biopsy, using an irradiated mouse fibroblast feeder layer and medium containing fetal bovine serum (FBS). However, the expansion of keratinocytes in vitro for clinical use has remained challenging, particularly as the use of undefined xenogeneic materials in the treatment of patients can be risky given the presence of xenopathogens. Existing in vitro culture systems that omit the feeder layer, serum, or animal original components have limited performance in culturing KSCs. For example, EpiLife from ThermoFisher (a sterile, liquid medium with 60 μM calcium chloride) with the Supplement S7 (a sterilized, concentrated, ionically balanced xeno-free solution) has been the golden commercial form. However, KSCs grown in this existing culture system have a more limited lifespan, with diminished self-renewal capacity and an increased commitment towards differentiation or senescence. Thus, there is a need for a new culture system that has an improved performance in expanding the primary KSCs long-term and/or retaining cell self-renewal and differentiation abilities in vitro.

### SUMMARY

Disclosed here are compositions and kits comprising a serum-free culture medium, as well as methods for making and using the culture medium.

In one aspect, disclosed is a serum-free composition, comprising: a coating matrix, a basal medium, and a growth supplement. In some cases, the composition is substantially free of human or non-human animal serum. In some cases, the composition is substantially free of cells or components derived from non-human animals. In some cases, the composition is animal component-free. In some cases, the composition is substantially free of mouse feeder layer, fetal bovine serum (FBS), bovine pituitary extract, or bovine serum albumin (BSA). In some cases, the coating matrix comprises at least one extracellular matrix protein or synthetic polymer. In some cases, the at least one extracellular matrix protein comprises collagen, elastin, fibronectin, laminin, vitronectin, gelatin, or any combination thereof. In some cases, the collagen comprises collagen type I, II, or IV. In some cases, the fibronectin comprises a recombinant human fibronectin (rh-fibronectin). In some cases, the laminin comprises a recombinant human laminin (rh-laminin). In some cases, the at least one synthetic polymer comprises poly-D-lysine, poly-L-ornithine or both. In some cases, the basal medium comprises glucose, amino acid, vitamin, sodium pyruvate, or any combination thereof. In some cases, the basal medium further comprises L-glutamine, L-leucine, L-lysine, magnesium chloride, magnesium sulfate, L-methionine, sodium bicarbonate, calcium chloride, hydrocortisone, isoprenaline chloride, liothyronine, adenine, L-Alanyl-L-Glutamine, insulin, transferrin, sodium selenite, or any combination thereof.

In some cases, the composition has a final calcium concentration of about 0.06 mM to about 1 mM. In some cases, the composition has a final calcium concentration of at least about 0.06 mM, about 0.08 mM, about 0.1 mM, about 0.15 mM, about 0.2 mM, about 0.5 mM, or about 1 mM. In some cases, the composition has a final calcium concentration of at least about 0.1 mM. In some cases, the composition has a final calcium concentration of at most about 1 mM. In some cases, the composition has a final calcium concentration of about 0.06 mM to about 0.08 mM, about 0.06 mM to about 0.1 mM, about 0.06 mM to about 0.15 mM, about 0.06 mM to about 0.2 mM, about 0.06 mM to about 0.5 mM, about 0.06 mM to about 1 mM, about 0.08 mM to about 0.1 mM, about 0.08 mM to about 0.15 mM, about 0.08 mM to about 0.2 mM, about 0.08 mM to about 0.5 mM, about 0.08 mM to about 1 mM, about 0.1 mM to about 0.15 mM, about 0.1 mM to about 0.2 mM, about 0.1 mM to about 0.5 mM, about 0.1 mM to about 1 mM, about 0.15 mM to about 0.2 mM, about 0.15 mM to about 0.5 mM, about 0.15 mM to about 1 mM, about 0.2 mM to about 0.5 mM, about 0.2 mM to about 1 mM or about 0.5 mM to about 1 mM. In some cases, the composition has a final calcium concentration of about 0.06 mM, about 0.08 mM, about 0.1 mM, about 0.15 mM, about 0.2 mM, about 0.5 mM, or about 1 mM. In some cases, the composition has a final calcium concentration of about 0.1-1.0 mM. In some cases, the composition has a final calcium concentration of about 0.15 mM.

In some cases, the growth supplement comprises at least one growth factor. In some cases, the at least one growth factor comprises epidermal growth factor (EGF), keratinocyte growth factor (KGF), or both. In some cases, the EGF comprises recombinant human epidermal growth factor (rh-EGF).

In some cases, the composition has a final EGF concentration of about 0.1 ng/mL to about 300 ng/mL. In some cases, the composition has a final EGF concentration of at least about 0.1 ng/mL. In some cases, the composition has a final EGF concentration of at least about 0.1 ng/mL, about 1 ng/mL, about 10 ng/mL, about 50 ng/mL, about 100 ng/mL, about 150 ng/mL, about 200 ng/mL, or about 300 ng/mL. In some cases, the composition has a final EGF concentration of at most about 300 ng/mL. In some cases, the composition has a final EGF concentration of about 0.1 ng/mL to about 1 ng/mL, about 0.1 ng/mL to about 10 ng/mL, about 0.1 ng/mL to about 50 ng/mL, about 0.1 ng/mL to about 100 ng/mL, about 0.1 ng/mL to about 150 ng/mL, about 0.1 ng/mL to about 200 ng/mL, about 0.1 ng/mL to about 300 ng/mL, about 1 ng/mL to about 10 ng/mL, about 1 ng/mL to about 50 ng/mL, about 1 ng/mL to about 100 ng/mL, about 1 ng/mL to about 150 ng/mL, about 1 ng/mL to about 200 ng/mL, about 1 ng/mL to about 300 ng/mL, about 10 ng/mL to about 50 ng/mL, about 10 ng/mL to about 100 ng/mL, about 10 ng/mL to about 150 ng/mL, about 10 ng/mL to about 200 ng/mL, about 10 ng/mL to about 300 ng/mL, about 50 ng/mL to about 100 ng/mL, about 50 ng/mL to about 150 ng/mL, about 50 ng/mL to about 200 ng/mL, about 50 ng/mL to about 300 ng/mL, about 100 ng/mL to about 150 ng/mL, about 100 ng/mL to about 200 ng/mL, about 100 ng/mL to about 300 ng/mL, about 150 ng/mL to about 200 ng/mL, about 150 ng/mL to about 300 ng/mL, or about 200 ng/mL to about 300 ng/mL. In some cases, the composition has a final EGF concentration of about 0.1 ng/mL, about 1 ng/mL, about 10 ng/mL, about 50 ng/mL, about 100 ng/mL, about 150 ng/mL, about 200 ng/mL, or about 300 ng/mL. In some cases, the composition has a final EGF concentration of about 10 ng/mL.

In some cases, the KGF comprises recombinant human keratinocyte growth factor (rh-KGF). In some cases, the composition has a final KGF concentration of about 0.1 ng/mL to about 300 ng/mL. In some cases, the composition has a final KGF concentration of at least about 0.1 ng/mL. In some cases, the composition has a final KGF concentration of at least about 0.1 ng/mL, about 1 ng/mL, about 10 ng/mL, about 50 ng/mL, about 100 ng/mL, about 150 ng/mL, about 200 ng/mL, or about 300 ng/mL. In some cases, the composition has a final KGF concentration of at most about 300 ng/mL. In some cases, the composition has a final KGF concentration of about 0.1 ng/mL to about 1 ng/mL, about 0.1 ng/mL to about 10 ng/mL, about 0.1 ng/mL to about 50 ng/mL, about 0.1 ng/mL to about 100 ng/mL, about 0.1 ng/mL to about 150 ng/mL, about 0.1 ng/mL to about 200 ng/mL, about 0.1 ng/mL to about 300 ng/mL, about 1 ng/mL to about 10 ng/mL, about 1 ng/mL to about 50 ng/mL, about 1 ng/mL to about 100 ng/mL, about 1 ng/mL to about 150 ng/mL, about 1 ng/mL to about 200 ng/mL, about 1 ng/mL to about 300 ng/mL, about 10 ng/mL to about 50 ng/mL, about 10 ng/mL to about 100 ng/mL, about 10 ng/mL to about 150 ng/mL, about 10 ng/mL to about 200 ng/mL, about 10 ng/mL to about 300 ng/mL, about 50 ng/mL to about 100 ng/mL, about 50 ng/mL to about 150 ng/mL, about 50 ng/mL to about 200 ng/mL, about 50 ng/mL to about 300 ng/mL, about 100 ng/mL to about 150 ng/mL, about 100 ng/mL to about 200 ng/mL, about 100 ng/mL to about 300 ng/mL, about 150 ng/mL to about 200 ng/mL, about 150 ng/mL to about 300 ng/mL, or about 200 ng/mL to about 300 ng/mL. In some cases, the composition has a final KGF concentration of about 0.1 ng/mL, about 1 ng/mL, about 10 ng/mL, about 50 ng/mL, about 100 ng/mL, about 150 ng/mL, about 200 ng/mL, or about 300 ng/mL. In some cases, the composition has a final KGF concentration of about 10 ng/mL.

In some cases, the growth supplement further comprises a rho kinase (ROCK) inhibitor, a transforming growth factor-beta (TGFB) receptor inhibitor, or both. In some cases, the ROCK inhibitor comprises AT-13148, BA-210, p3-elemene, DJ4, fasudil, GSK-576371, GSK429286A, H-1152, hydroxyfasudil, ibuprofen, LX-7101, netarsudil, RKI-1447, ripasudil, TCS-7001, thiazovivin, verosudil, Y-27632, Y-30141, Y-33075, Y-39983, or any combination thereof. In some cases, the composition has a final ROCK inhibitor concentration of about 0.1 μM to about 300 μM. In some cases, the composition has a final ROCK inhibitor concentration of at least about 0.1 μM. In some cases, the composition has a final ROCK inhibitor concentration of at least about 0.1 μM, about 1 μM, about 10 μM, about 20 μM, about 50 μM, about 100 μM, about 150 μM, about 200 μM, or about 300 μM. In some cases, the composition has a final ROCK inhibitor concentration of at most about 300 μM. In some cases, the composition has a final ROCK inhibitor concentration of about 0.1 μM to about 1 μM, about 0.1 μM to about 10 μM, about 0.1 μM to about 20 μM, about 0.1 μM to about 50 μM, about 0.1 μM to about 100 μM, about 0.1 μM to about 150 μM, about 0.1 μM to about 200 μM, about 0.1 μM to about 300 μM, about 1 μM to about 10 μM, about 1 μM to about 20 μM, about 1 μM to about 50 μM, about 1 μM to about 100 μM, about 1 μM to about 150 μM, about 1 μM to about 200 μM, about 1 μM to about 300 μM, about 10 μM to about 20 μM, about 10 μM to about 50 μM, about 10 μM to about 100 μM, about 10 μM to about 150 μM, about 10 μM to about 200 μM, about 10 μM to about 300 μM, about 20 μM to about 50 μM, about 20 μM to about 100 μM, about 20 μM to about 150 μM, about 20 μM to about 200 μM, about 20 μM to about 300 μM, about 50 μM to about 100 μM, about 50 μM to about 150 μM, about 50 μM to about 200 μM, about 50 μM to about 300 μM, about 100 μM to about 150 μM, about 100 μM to about 200 μM, about 100 μM to about 300 μM, about 150 μM to about 200 μM, about 150 μM to about 300 μM, or about 200 μM to about 300 μM. In some cases, the composition has a final ROCK inhibitor concentration of about 0.1 μM, about 1 μM, about 10 μM, about 20 μM, about 50 μM, about 100 μM, about 150 μM, about 200 μM, or about 300 μM. In some cases, the composition has a final ROCK inhibitor concentration of about 0.1 μM-100 μM. In some cases, the composition has a final ROCK inhibitor concentration of about 10 μM.

In some cases, the TGFB receptor inhibitor comprises RepSox, SB-431542, A83-01, Galunisertib, or any combination thereof. In some cases, the composition has a final TGFB inhibitor concentration of about 0.01 μM to about 10 μM. In some cases, the composition has a final TGFB inhibitor concentration of at least about 0.01 μM. In some cases, the composition has a final TGFB inhibitor concentration of at least about 0.01 μM, about 0.05 μM, about 0.1 μM, about 0.2 μM, about 0.5 μM, about 1 μM, about 2 μM, about 5 μM, or about 10 μM. In some cases, the composition has a final TGFB inhibitor concentration of at most about 10 μM. In some cases, the composition has a final TGFB inhibitor concentration of about 0.01 μM to about 0.05 μM, about 0.01 μM to about 0.1 μM, about 0.01 μM to about 0.2 μM, about 0.01 μM to about 0.5 μM, about 0.01 μM to about 1 μM, about 0.01 μM to about 2 μM, about 0.01 μM to about 5 μM, about 0.01 μM to about 10 μM, about 0.05 μM to about 0.1 μM, about 0.05 μM to about 0.2 μM, about 0.05 μM to about 0.5 μM, about 0.05 μM to about 1 μM, about 0.05 μM to about 2 μM, about 0.05 μM to about 5 μM, about 0.05 μM to about 10 μM, about 0.1 μM to about 0.2 μM, about 0.1 μM to about 0.5 μM, about 0.1 μM to about 1 μM, about 0.1 μM to about 2 μM, about 0.1 μM to about 5 μM, about 0.1 μM to about 10 μM, about 0.2 μM to about 0.5 μM, about 0.2 μM to about 1 μM, about 0.2 μM to about 2 μM, about 0.2 μM to about 5 μM, about 0.2 μM to about 10 μM, about 0.5 μM to about 1 μM, about 0.5 μM to about 2 μM, about 0.5 μM to about 5 μM, about 0.5 μM to about 10 μM, about 1 μM to about 2 μM, about 1 μM to about 5 μM, about 1 μM to about 10 μM, about 2 μM to about 5 μM, about 2 μM to about 10 μM, or about 5 μM to about 10 μM. In some cases, the composition has a final TGFB inhibitor concentration of about 0.01 μM, about 0.05 μM, about 0.1 μM, about 0.2 μM, about 0.5 μM, about 1 μM, about 2 μM, about 5 μM, or about 10 μM. In some cases, the composition has a final TGFB receptor inhibitor concentration of about 0.5 μM.

In another aspect, disclosed here is a method of preparing the serum-free composition previously described above, comprising: a) mixing the basal medium and growth supplement to form a mixture solution; and b) contacting the coating matrix with the mixture solution.

In another aspect, disclosed here is a method of culturing a plurality of cells in the serum-free composition previously described above, comprising: contacting the serum-free composition with the plurality of cells. In some cases, the plurality of cells comprise stem cells. In some cases, the plurality of cells comprise keratinocyte stem cells (KSCs). In some cases, the KSCs comprise adult KSCs. In some cases, the KSCs comprise fetal KSCs. In some cases, the plurality of cells comprise corneal epithelial stem cells.

In another aspect, disclosed here is a kit, comprising a coating matrix, a basal medium, and a concentrated stock of a growth supplement. In some cases, the coating matrix is substantially free of mouse feeder layer. In some cases, the basal medium is substantially free of human or non-human animal serum. In some cases, the coating matrix, basal medium, and growth supplement are substantially free of cells or components derived from non-human animals. In some cases, the coating matrix, basal medium, and growth supplement is substantially free of mouse feeder layer, fetal bovine serum (FBS), bovine pituitary extract, or bovine serum albumin (BSA). In some cases, the coating matrix comprises at least one extracellular matrix protein or synthetic polymer. In some cases, the at least one extracellular matrix protein comprises collagen, elastin, fibronectin, laminin, vitronectin, gelatin, or any combination thereof. In some cases, the collagen comprises collagen type I, II, or IV. In some cases, the fibronectin comprises a recombinant human fibronectin (rh-fibronectin). In some cases, the laminin comprises a recombinant human laminin (rh-laminin). In some cases, the at least one synthetic polymer comprises poly-D-lysine, poly-L-ornithine or both. In some cases, the basal medium comprises glucose, amino acid, vitamin, sodium pyruvate, or any combination thereof. In some cases, the basal medium further comprises L-glutamine, L-leucine, L-lysine, magnesium chloride, magnesium sulfate, L-methionine, sodium bicarbonate, calcium chloride, hydrocortisone, isoprenaline chloride, liothyronine, adenine, L-Alanyl-L-Glutamine, insulin, transferrin, sodium selenite, or any combination thereof. In some cases, the basal medium has a final calcium concentration of at least about 0.1 mM. In some cases, the basal medium has a final calcium concentration of about 0.1-1.0 mM. In some cases, the basal medium has a final calcium concentration of about 0.15 mM. In some cases, the growth supplement comprises at least one growth factor. In some cases, the at least one growth factor comprises epidermal growth factor (EGF), keratinocyte growth factor (KGF), or both. In some cases, the EGF comprises recombinant human epidermal growth factor (rh-EGF). In some cases, the concentrated stock of the growth supplement has a final EGF concentration of about 0.1-300 μg/mL. In some cases, the concentrated stock of the growth supplement has a final EGF concentration of about 10 μg/mL. In some cases, the KGF comprises recombinant human keratinocyte growth factor (rh-KGF). In some cases, the concentrated stock of the growth supplement has a final KGF concentration of about 0.1-300 μg/mL. In some cases, the concentrated stock of the growth supplement has a final KGF concentration of about 10 μg/mL. In some cases, the growth supplement further comprises a rho kinase (ROCK) inhibitor, a transforming growth factor-beta (TGFB) receptor inhibitor, or both. In some cases, the ROCK inhibitor comprises AT-13148, BA-210, p3-elemene, DJ4, fasudil, GSK-576371, GSK429286A, H-1152, hydroxyfasudil, ibuprofen, LX-7101, netarsudil, RKI-1447, ripasudil, TCS-7001, thiazovivin, verosudil, Y-27632, Y-30141, Y-33075, Y-39983, or any combination thereof.

In some cases, the concentrated stock of the growth supplement has a final ROCK inhibitor concentration of about 0.1-100 mM. In some cases, the concentrated stock of the growth supplement has a final ROCK inhibitor concentration of about 10 mM. In some cases, the TGFB receptor inhibitor comprises RepSox, SB-431542, A83-01, Galunisertib, or any combination thereof. In some cases, the composition has a final TGFB receptor inhibitor concentration of about 0.01-10 mM. In some cases, the composition has a final TGFB receptor inhibitor concentration of about 0.5 mM.

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows an exemplary diagram of skin structure;

FIG. 2 shows adult KSCs on different coating matrix after 30 min of culturing;

FIG. 3 shows adult KSCs on different coating matrix after 2 hours of culturing;

FIG. 4 shows adult KSCs on different coating matrix after 6 hours of culturing;

FIG. 5 shows adult KSCs on different coating matrix after 24 hours of culturing;

FIG. 6 shows the population doubling levels of adult KSCs cultured on different coating matrix as well as blank for 5 passages;

FIG. 7 shows the average of viability of culturing adult KSCs on different coating matrix for 5 passages;

FIG. 8 shows the population doubling levels of adult KSCs cultured in different concentrations of calcium for 10 passages;

FIGS. 9A-9E show adult KSCs at the final passage for each condition of different concentrations of calcium, including A: 0, B: 1.5 mM, C: 0.5 mM, D: 0.05 mM, E: 0.15 mM;

FIG. 10 shows the function of Y-27632 of adult KSCs proliferation;

FIG. 11 shows the function of A 83-01 of adult KSCs proliferation;

FIG. 12 shows the function of EGF of adult KSCs proliferation;

FIG. 13 shows the function of KGF of adult KSCs proliferation;

FIG. 14 shows the population doubling levels of adult KSCs cultured in KSFX-free basal, complete, and EpiLife;

FIG. 15 shows the population doubling levels of adult KSCs from a second donor cultured in KSFX-free basal, complete, and EpiLife;

FIG. 16 shows the expression of KRT14 in adult KSCs cultured in KSFX-free complete medium and EpiLfe;

FIG. 17 shows the population doubling levels of fetal KSCs cultured in KSFX-free complete medium and EpiLife;

FIG. 18 shows the population doubling levels of corneal epithelial stem cells cultured in KSFX-free complete medium.

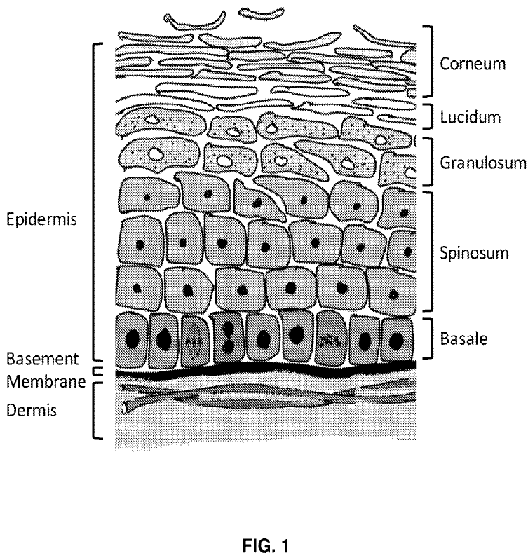

### DETAILED DESCRIPTION

Disclosed here are compositions and kits comprising a serum-free culture medium, as well as methods for making and using the culture medium. In some cases, the culture medium is a serum-free, feeder-free, and xeno-free culture system (Epi-Zero) that includes a coating matrix, a basal medium, and a growth supplement. In some cases, the culture system can long-term expand stem cells such as the primary KSCs and/or retain their self-renewal and differentiation abilities in vitro. In some cases, the culture system can support the constant proliferation of KSCs and/or maintain the KSCs cell-specificity, such as the expression of KRT14.

Because the cells are not cultivated in the presence of animal components, the risk of cross-species contamination in clinical applications can be eliminated.

### Definitions

The term “about” and its grammatical equivalents in relation to a reference numerical value and its grammatical equivalents as used herein can include a range of values plus or minus 10% from that value, such as a range of values plus or minus 10%, 9%, 8%, 7%, 6%, 5%, 4%, 3%, 2%, or 1% from that value. For example, the amount “about 10” includes amounts from 9 to 11.

Unless otherwise indicated, some embodiments herein contemplate numerical ranges. When a numerical range is provided, unless otherwise indicated, the range includes the range endpoints. Unless otherwise indicated, numerical ranges include all values and sub ranges therein as if explicitly written out.

The singular forms “a,” “an,” and “the” are used herein to include plural references and vice versa unless the context clearly dictates otherwise. Accordingly, unless the contrary is indicated, the numerical parameters set forth in this application are approximations that may vary depending upon the desired properties sought to be obtained by the present invention.

Unless otherwise indicated, open terms for example “contain,” “containing,” “include,” “including,” and the like mean comprising.

The term “stem cell” refers to undifferentiated or partially differentiated cell that can differentiate into at least another type of cell and/or proliferate long term to produce more of the same stem cell. For example, a stem cell can be a keratinocyte stem cell (KSC) or a corneal epithelial stem cell.

The term “basal medium” refers to any medium which is capable of supporting growth of cells (e.g., KSCs), when supplemented either with serum or with the serum-free cell culture of the present invention. The basal medium can supply standard inorganic salts, such as zinc, iron, magnesium, calcium and potassium, as well as trace elements, vitamins, an energy source, a buffer system, and essential amino acids.

The term “serum-free composition” or “serum-free” refers to a composition that does not contain serum, plasma, or hemolymph as an ingredient.

The term “animal component-free” or “animal origin-free” refers to a composition that does not contain any ingredient that is either a non-human animal tissue or body fluid or that is isolated or purified from a non-human animal tissue or body fluid.

The term “inhibitor” refers to agents that, e.g., inhibit expression or bind to, partially or totally block stimulation or protease inhibitor activity, decrease, prevent, delay activation, inactivate, desensitize, or down regulate the activity of the described target protein, e.g., antagonists. Assays for inhibitors include, e.g., applying putative modulator compounds to cells expressing the described target protein and then determining the functional effects on the described target protein activity, as described above. Samples or assays comprising described target protein that are treated with a potential inhibitor are compared to control samples without the inhibitor to examine the extent of effect. Control samples (untreated) are assigned a relative activity value of 100%. Inhibition of a described target protein is achieved when the activity value relative to the control is about 80%, optionally 50% or 25, 10%, 5% or 1%.

Unless defined otherwise, all technical and scientific terms used herein have the same meaning as commonly understood by one of the ordinary skill in the art to which this invention belongs. Although any methods and materials similar or equivalent to those described herein can be used in the practice or testing of the formulations or unit doses herein, some methods and materials are now described. Unless mentioned otherwise, the techniques employed or contemplated herein are standard methodologies. The materials, methods and examples are illustrative only and not limiting.

### Coating Matrix

Disclosed here is a serum-free composition comprising a coating matrix. Extracellular matrix (ECM) is a complex meshwork of macromolecules, comprising fibrous structural proteins (e.g., collagen, fibronectin, laminin, and elastin), specialized proteins (e.g., growth factors), and/or proteoglycans (e.g., perlecan). It can provide a platform for cell adhesion and can also provide both biochemical and biomechanical cues that regulate cell behaviors like adhesion, migration, proliferation, and/or differentiation. The basement membrane (BM) is a thin, pliable sheet-like type of ECM, containing collagen, laminin, heparan sulfate, entactin, and/or fibronectin. It can provide both structural support to cultured cells such as keratinocyte stem cells (KSCs) or corneal epithelial stem cells, and can mediate the signals that influencing cell behaviors.

One common source of ECM is Matrigel (MAT), which is solubilized basement membrane preparation usually extracted from the mouse sarcoma, rich in extracellular matrix proteins including laminin, collagen, heparan sulfate proteoglycans, entactin/nidogen, and some growth factors. However, the disadvantages associated with MAT are commonly occurring lot-to-lot variability during the manufacturing and the complexity in composition, which is often ill-defined, making it difficult to determine exactly which signals are promoting the cell function. Collagen I (COLI) is also used either as a thin layer on tissue culture surfaces to enhance cell attachment and proliferation, or as a gel to promote the expression of cell-specific morphology and function. Recombinant Collagen I can be very expensive and/or lacks bioactivity, the reagent for research is usually extracted from the rat tails, which may cause xenogeneic problems for the clinical applications. Poly-D-Lysine (PDL) is a synthetic molecule that can be used as a thin coating to enhance the attachment of cells to plastic and glass surfaces. Fibronectin (FN) can be also used to promote attachment, spreading, and proliferation of cells. The principal functions of FN appear to be in cellular migration during wound healing and development, regulation of cell growth and differentiation, and hemostasis. Laminin (LN) is a major component of basement membranes and can be used for the promotion of cell adhesion, migration, chemotaxis, growth, and/or differentiation, including neurite outgrowth.

### Basal Medium

Disclosed here is a serum-free composition comprising a basal medium.

The basal medium may include amino acids, vitamins, inorganic salts, sugars, buffering salts, lipids, insulin (or insulin substitute) and/or transferrin (or transferrin substitute). In some cases, the basal media may contain L-glutamine, L-leucine, L-lysine, magnesium chloride, magnesium sulfate, L-methionine, sodium bicarbonate, calcium chloride, hydrocortisone, isoprenaline chloride, liothyronine, adenine, L-Alanyl-L-Glutamine, insulin, transferrin, and sodium selenite. In some cases, the basal medium contains no serum.

Transferrin may be in the iron-free form (i.e., apotransferrin) or in the iron-complexed form (i.e., ferrotransferrin or holotransferrin). Insulin, if present, may be human-derived or recombinant. Transferrin may be replaced by ferric citrate or ferrous sulfate. Insulin may be replaced by one or more zinc-containing compounds such alone or more zinc salts. Zinc-containing compounds which may be used include but are not limited to ZnCl, Zn(NO₃)₂, ZnBr, and ZnSO₄, any of which may be present in their anhydrous or hydrated (i.e., “H₂O”) forms.

Amino acid ingredients may include but not limited to L-alanine, L-arginine, L-asparagine, L-aspartic acid, L-cysteine, L-glutamic acid, L-glutamine, glycine, L-histidine, L-isoleucine, L-leucine, L-lysine, L-methionine, L-phenylalanine, L-proline, L-serine, L-threonine, L-tryptophan, L-tyrosine and L-valine.

Vitamin ingredients may include but not limited to biotin, choline chloride, D-Ca++-pantothenate, folic acid, i-inositol, niacinamide, pyridoxine, riboflavin, thiamine and vitamin B12.

Inorganic salt ingredients may include but not limited to one or more calcium salts (e.g., CaCl₂)), Fe(NO₃)₃, KCl, one or more magnesium salts (e.g., MgCl₂ and/or MgSO₄), one or more manganese salts (e.g., MnCl₂), NaCl, NaHCO₃, Na₂HPO₄, and ions of the trace elements selenium, vanadium and zinc.

Calcium can be a regulator of keratinocytes or corneal epithelial cells differentiation in vivo and in vitro. A calcium gradient within the epidermis can promote the sequential differentiation of keratinocytes, as they traverse the different layers of the epidermis to form the permeability barrier of the stratum corneum. A number of signaling pathways involved with KSCs differentiation can be regulated by calcium, including the formation of desmosomes, adherens junctions, and tight junctions, which maintain the cell-cell adhesion and play an important intracellular signaling role through the activation of various kinases and phospholipases. In some cases, the serum-free composition has a final calcium concentration of about 0.06 mM to about 1 mM. In some cases, the composition has a final calcium concentration of at least about 0.1 mM. In some cases, the composition has a final calcium concentration of at least about 0.06 mM. In some cases, the composition has a final calcium concentration of at least about 0.06 mM, about 0.08 mM, about 0.1 mM, about 0.15 mM, about 0.2 mM, about 0.5 mM, or about 1 mM. In some cases, the composition has a final calcium concentration of at most about 1 mM. In some cases, the composition has a final calcium concentration of about 0.06 mM to about 0.08 mM, about 0.06 mM to about 0.1 mM, about 0.06 mM to about 0.15 mM, about 0.06 mM to about 0.2 mM, about 0.06 mM to about 0.5 mM, about 0.06 mM to about 1 mM, about 0.08 mM to about 0.1 mM, about 0.08 mM to about 0.15 mM, about 0.08 mM to about 0.2 mM, about 0.08 mM to about 0.5 mM, about 0.08 mM to about 1 mM, about 0.1 mM to about 0.15 mM, about 0.1 mM to about 0.2 mM, about 0.1 mM to about 0.5 mM, about 0.1 mM to about 1 mM, about 0.15 mM to about 0.2 mM, about 0.15 mM to about 0.5 mM, about 0.15 mM to about 1 mM, about 0.2 mM to about 0.5 mM, about 0.2 mM to about 1 mM, or about 0.5 mM to about 1 mM. In some cases, the composition has a final calcium concentration of about 0.06 mM, about 0.08 mM, about 0.1 mM, about 0.15 mM, about 0.2 mM, about 0.5 mM, or about 1 mM. In some cases, the composition has a final calcium concentration of about 0.1-1.0 mM. In some cases, the composition has a final calcium concentration of about 0.15 mM.

### Growth Supplement

Disclosed here is a serum-free composition comprising a growth supplement. KSCs' self-renewal and differentiation can be regulated by specific signaling pathways such as Rho and ROCK, TGF-β, EGFR, and KGF32-41. The Rho family of small GTP-binding proteins can affect a wide range of cell functions, such as assembly of the actin cytoskeleton, cell proliferation, and motility. Two Rho associated protein kinases (ROCKs) in the mammalian system are downstream effectors of Rho, and their activation is correlated with the stress-fiber formation and cellular contraction. Y-27632 can inhibit both ROCK isoforms and/or prevent dissociation-induced apoptosis in human embryonic stem cells as well as in keratinocytes and corneal epithelial cells. Transforming growth factor can causes normal human KSCs to arrest growth predominantly in the G1 phase of the cell cycle in a serum-free medium. Inhibition of TGF-β signaling activity in KSCs can prolong the culturing in vitro. A 83-01 is a potent inhibitor of activin receptor-like kinase (ALK) including ALK5, ALK4, and ALK7. In addition, EGF and KGF can promote the proliferation of KSCs in vitro.

The growth supplement may include one or more cytokines (e.g., growth factors such as EGF, aFGF, bFGF, IGF-1, IGF-2, HB-EGF, KGF, HGF, and the like), heparin (to stabilize heparin-binding growth factors such as the FGFs, HB-EGF, KGF and HGF) and one or more peptides derived from yeasts (e.g., yeast extract, yeastolate or yeast extract ultrafiltrate) or plants (e.g., rice or soy peptides).

### Complete Medium

The complete medium can be dissolved in a liquid carrier or maintained in dry form. If dissolved in a liquid carrier, the pH of the medium can be adjusted to about 7.0-7.6, preferably about 7.1-7.5, and most preferably about 7.2-7.4. The osmolarity of the medium should also be adjusted to about 260 mOsm to about 300 mOsm, preferably about 265 mOsm to about 280 mOsm, and most preferably about 265 mOsm to about 275 mOsm.

The solutions comprising individual ingredients can be more concentrated than the concentration of the same ingredients in a 1× media formulation. The ingredients can be 10-fold more concentrated (10× formulation), 25-fold more concentrated (25× formulation), 50-fold more concentrated (50× concentration), or 100-fold more concentrated (100× formulation). More highly concentrated formulations can be made, provided that the ingredients remain soluble and stable. If the individual medium ingredients are prepared as separate concentrated solutions, an appropriate (sufficient) amount of each concentrate can be combined with a diluent to produce a 1× medium formulation. Typically, the diluent used is water but other solutions including aqueous buffers, aqueous saline solution, or other aqueous solutions may be used according to the invention.

The culture media of the present invention can be sterilized to prevent unwanted contamination. Sterilization may be accomplished, for example, by filtration through a low protein-binding membrane filter of about 0.22 μm or about 0.45 μm pore size after admixing the concentrated ingredients to produce a sterile culture medium. Alternatively, concentrated subgroups of ingredients may be filter-sterilized and stored as sterile solutions. These sterile concentrates can then be mixed under aseptic conditions with a sterile diluent to produce a concentrated 1× sterile medium formulation.

### Examples

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Example 1—Preparation of the Serum-Free Composition

Coating Matrix Preparation—Coating matrix such as Collagen I, Poly-D-Lysine (PDL), Matrigel, rh-Fibronectin, and rh-Laminin 521 were prepared according to the instructions of the manufacture (Table 1). Briefly, for one well of the 6-well cell culture plate, 1 mL of the working solution was added to the surface of the well and the plate was incubated at the recommended condition. Then the well was washed once with 1 mL of PBS and ready to be used.

TABLE 1
Summary of the coating matrix.

| Coating | Working | Incubation | Resource | Resource |
|---|---|---|---|---|
| Matrix | Concentration | Condition | Xeno-free | Xeno-free |
| PDL | 50 μg/mL | RT, 1 hr | Synthetic | Yes |
| Fibronectin | 10 μg/mL | RT, 1 hr | Oryza sativa | Yes |
| | | | recombinant | |
| Laminin | 10 μg/mL | 37 C., 2 | Human cell line | Yes |
| | | hrs | recombinant | |
| Collagen I | 50 μg/mL | RT, 1 hr | Extracted from | No |
| | | | rat tail | |
| Matrigel | 100 μg/mL | RT, 1 hr | Extracted from | No |
| | | | mouse sarcoma | |

Basal Medium Preparation—The serum/feeder/xeno-free (KSFX-free) basal medium was formulated to contain 14.8 g of Dulbecco's Modified Eagle Medium (DMEM)/F12 powder per liter of medium, or alternatively, a 1:1 mixture of DMEM and F12 with other supplements, including 0.365 g/L L-glutamine, 0.05905 g/L L-leucine, 0.09125 g/L L-lysine, 0.0612 g/L magnesium chloride, 0.04884 g/L magnesium sulfate, 0.0172 g/L L-methionine, 1.2 g/L sodium bicarbonate, 0.01661 g/L calcium chloride, 0.4 mg/L hydrocortisone, 1.98 mg/L isoprenaline chloride, 0.0014 mg/L liothyronine, 24 mg/L adenine, 0.434 g/L L-Alanyl-L-Glutamine, 10 mg/L insulin, 5.5 mg/L transferrin, and 0.0067 mg/L sodium selenite.

Growth Supplement Preparation—A growth supplement (1000×) was formulated to contain ROCK inhibitor 10 mM of Y-27632, TGFB inhibitor 0.5 mM of A83-01, rh-EGF 10 μg/mL, and rhKGF 10 μg/mL. To make the serum/feeder/xeno-free complete medium, add 1 mL of the growth supplement to 1 L of the basal medium and mix thoroughly.

Control Medium Preparation—To make a complete EpiLife culture medium, add 5 mL of the Supplement S7 to 500 mL of the EpiLife liquid medium and mix thoroughly.

### Example 2—KSCs Isolation and Culture In Vitro

The keratinocyte stem cells (KSCs) were isolated from donated foreskin or fetal skin tissue. Briefly, the skin tissue was cleaned up with 70% ethanol and washed several times with PBS containing antibiotics. The tissue in PBS was collected, trimmed away any fat and loose fascia, and cut into strips approximately 0.5 cm×1.5 cm. The tissue was submerged in dispase and treated overnight at 4° C. The epidermis was then separated from the dermis and washed with PBS several times. All the epidermis was collected into one tube containing the TrypLE and incubated for 30 minutes at 37° C. Enough PBS was added to neutralize the TrypLE and the solution was pipetted up and down to suspend the cells. The cell suspension was passed through a 70 μm cell strainer, and then the cells were pelleted by centrifugation at 300 g for 10 minutes. The cell pellet was resuspended in culture medium and the concentration of the viable cells/mL was determined. 2×10⁴ viable epidermal cells/cm² was plated on culture flask treated with coating matrix, and then the flask was incubated in a 37° C., 95% air/5% CO₂ saturated humidity chamber. The culture medium was changed every 48 hours. Once the cultures were ˜80% confluent, the cells were subcultured and/or cryopreserved using a freezing medium. For subculture, cells were washed once by PBS then treated with TrypLE for no more than 6 min at 37° C. PBS was added to neutralize the TrypLE, and then the tube was centrifuged at 300 g for 10 min. New flasks were seeded at 1-2×10⁴ cells/cm².

### Example 3—Cell Proliferation Assay

For long-term proliferation assay, cells were continuously passaged on the 6-well cell culture plate. For each passage, 2×10⁵ viable cells were plated and then cultured for about 5 days, when the cells were ˜90% confluent, cells were passaged and the cell number as well as the viability with the cell counter were recorded. Microscopy was performed with Eclipse TS100 (Nikon).

For the EdU (5-ethynyl-2′-deoxyuridine) assay, 5×10³ cells were plated to a well of the 96-well cell culture plate and cultured for overnight. On the second day, the cells were treated with 10 μM EdU for 3 hours at 37° C. The cells were washed with PBS and then fixed with 4% formaldehyde for 30 min at RT. The cells were washed with PBS and then permeabilized with 0.1% Triton for 20 min at RT. The cells were washed with PBS and then labeled with freshly prepared EdU labeling buffer containing 5 μM of Sulfo-Cyanine 3 azide, 4 mM of Copper(II) sulfate pentahydrate, 2 mg/mL of Sodium L-ascorbate in PBS for 30 min at RT. The cells were washed with PBS and labeled with Hoechst if needed. Finally, the plate was scanned and analyzed by Celllnsight CX5 HCA reader.

### Example 4—Immunofluorescence

Cells were washed with PBS once and then fixed with 4% formaldehyde for 30 min at room temperature (RT). The cells were washed with PBS and then permeabilized with 0.1% Triton for 20 min at RT The cells were washed with PBS and then blocked with 3% BSA for 1 hr at RT. Then the cells were incubated with primary antibodies such as anti-KRT14, or anti-p63 overnight at 4° C. The cells were washed with PBS and labeled with the secondary antibody such as Goat anti-Mouse Alexa Fluor 488, Goat anti-Rabbit Alexa Fluor 647, and Hoechst if needed. Finally, the plate was scanned and analyzed by CellInsight CX5 HCA reader. Microscopy was captured with Confocal Microscope A1 HD25.

### Example 5—Coating Matrix Function Assay

The function of each coating matrix listed in Table 1 was tested for the attachment and spreading of adult KSCs. Cells were plated on the different coating matrix and cultured in the KSFX-free basal medium for 24 hours. Bright-field microscopy was performed at different time points.

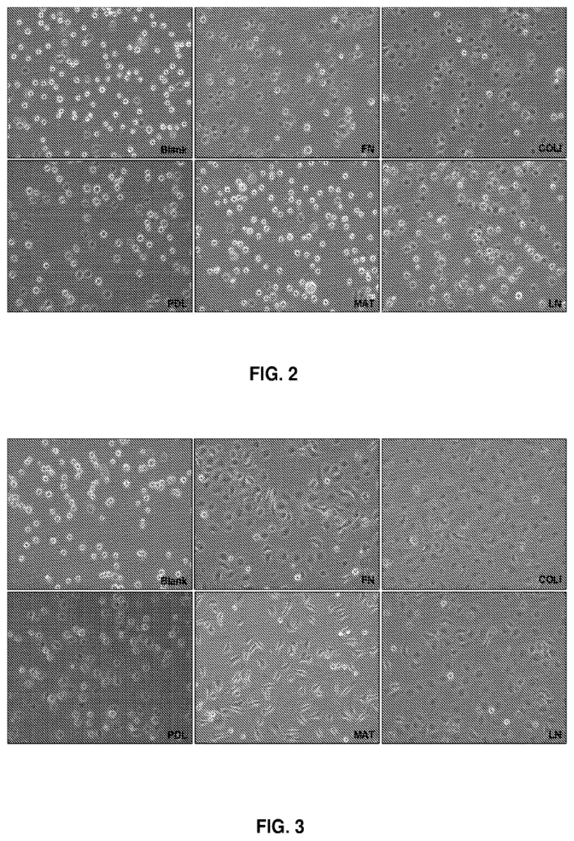

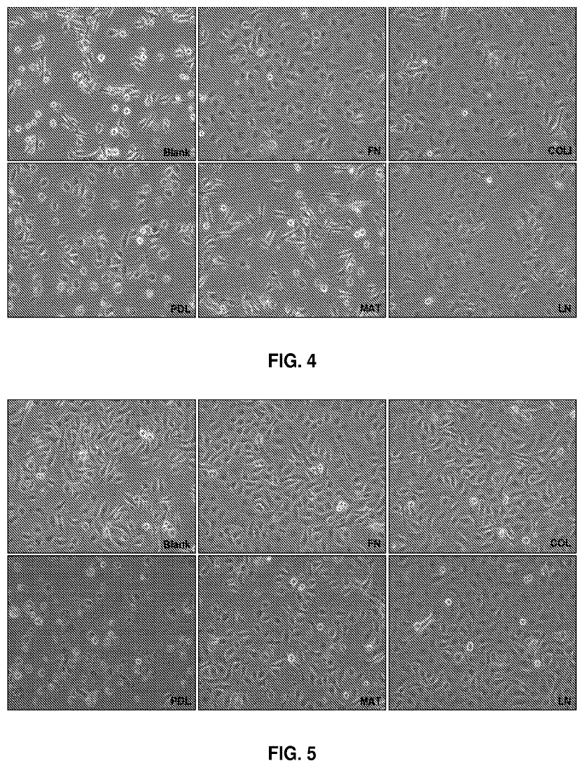

As shown in FIG. 2, after 30 min of culturing, adult KSCs on FN, COLI, and LN had attached to the plate and spread with a flatten morphology. Cells on PDL were attached to the plate but not spread yet, while cells in blank and on MAT were still suspending with a round morphology. As shown in FIG. 3, after 2 hours of culturing, adult KSCs on FN, COLI, and LN had already fully spread but not for cells on PDL. Cells on MAT had spread but with a fibroblast-like morphology, while cells in blank were still suspending. After 6 hours of culturing, adult KSCs in blank spread, showing a similar fibroblast-like morphology with cells on MAT, while cells on PDL still did not spread very well (FIG. 4). As shown in FIG. 5, after 24 hours of culturing, all the conditions, except for the PDL, had shown the fully spreading of adult KSCs. Blebbing was also observed in the cells cultured on PDL, which indicates the apoptosis of adult KSCs on PDL. Based on the attachment/spreading function assay, the coating matrix can promote the attachment as well as the spreading of adult KSCs cultured in the KSFX-free basal medium. Among all the coating matrix tested, FN, COLI, and LN performed better functions compared with MAT and PDL.

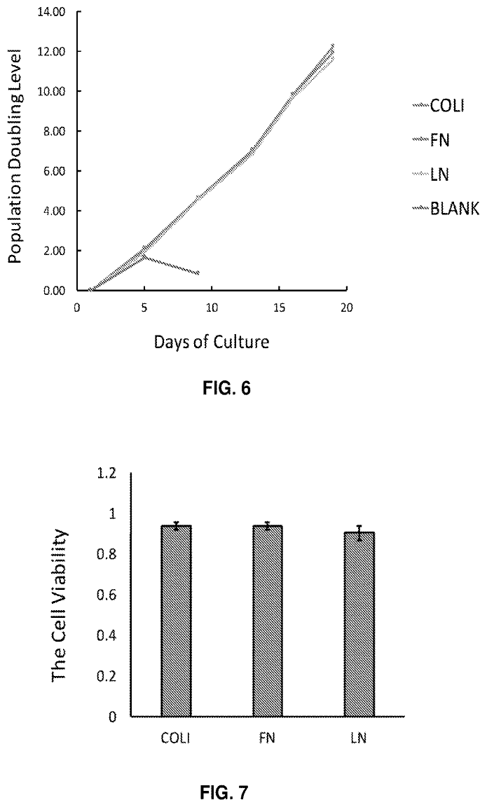

Further, the proliferation functions of FN, COLI, and LN was also tested in the KSFX-free basal medium. Adult KSCs were continuously passaged and the population doubling (PD) curves were calculated. Compared with blank, coating matrix, including FN, COLI, and LN could promote the proliferation of adult KSCs (FIG. 6) and supported the long-term expansion of adult KSCs in vitro (FIG. 7).

### Example 6—Calcium Concentration Function Assay

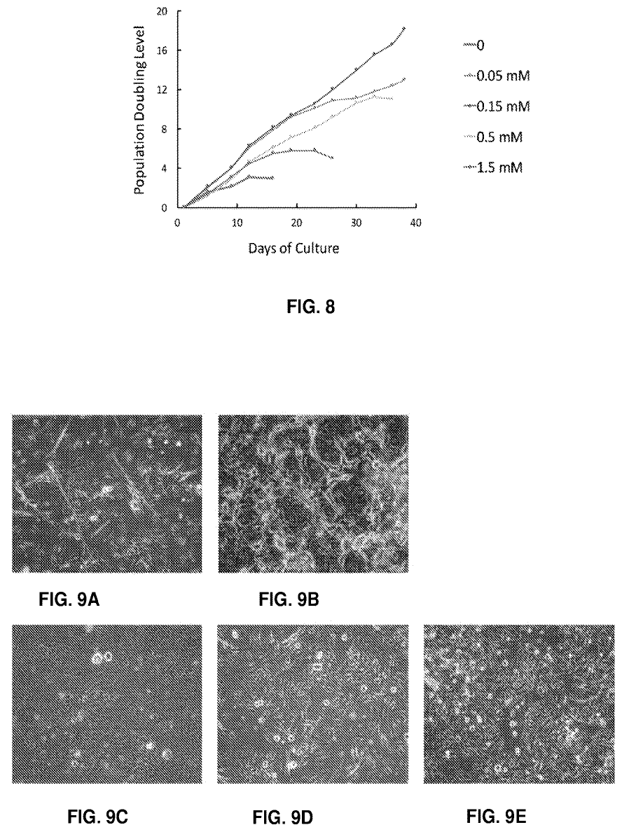

The effect of calcium concentrations in the KSFX-free basal medium on the proliferation and differentiation of adult KSCs were tested. Cells were cultured in different concentrations of calcium, including 0 mM, 0.05 mM, 0.15 mM, 0.5 mM, and 1.5 mM for 10 passages. As shown in FIG. 8 and FIG. 9A, withdraw of calcium led to the growth stop of adult KSCs after 3 passages. Adult KSCs cultured in 1.5 mM of calcium stopped growing after 5 passages and most of cells had differentiated with the collapsed nucleus (FIG. 8 and FIG. 9B). Adult KSCs cultured in 0.5 mM of calcium could proliferate longer, with more cell-to-cell connections, which could induce epithelial cadherins (E-cadherin) to mediates the cell-cell adhesions (FIG. 8 and FIG. 9C). Adult KSCs cultured in 0.05 mM and 0.15 mM presented the similar proliferation rate at the early passage. Cells in 0.05 mM started to grow slower later (FIG. 8, FIG. 9D, and FIG. 9E).

The result of this experiment shows that the appropriate concentration of calcium can impact the long-term expansion of adult KSCs in vitro. Calcium concentrations that are too-low (e.g., 0 mM) can stop proliferation of the KSCs, while high concentrations (e.g., 1.5 mM) can cause over differentiation of the KSCs. Intermediate concentrations such as 0.1-1.0 mM (e.g., 0.15 mM) of calcium can better support the long-term expansion of adult KSCs in vitro.

### Example 7—Growth Supplement Function Assay

Though some inhibitors or growth factors have been studied for their function in KSCs traditional culturing systems, their function has not been tested in a serum-free, feeder-free, and xeno-free culture condition.

Here, the function of a rho kinase (ROCK) inhibitor (Y-27632), a transforming growth factor-beta (TGFB) receptor inhibitor (A 83-01), an epidermal growth factor (rh-EGF), and a keratinocyte growth factor (rh-KGF) were tested in adult KSCs' proliferation using the EdU assay. 5-Ethynyl-2′-deoxyuridine (EdU) is a thymidine analogue, which can be used to assay DNA synthesis in cell culture and detect cells in embryonic, neonatal and adult animals which have undergone DNA synthesis.

Different concentrations of each factor were added to the KSFX-free basal medium and the adult KSCs were cultured for 24 hours. Then cells were labeled with EdU as well as the KSCs marker p63. The ratio of EdU/p63 indicates the proliferation rate of KSCs, and the p63 count indicates the total cell number which is a result of cell proliferation and cell death.

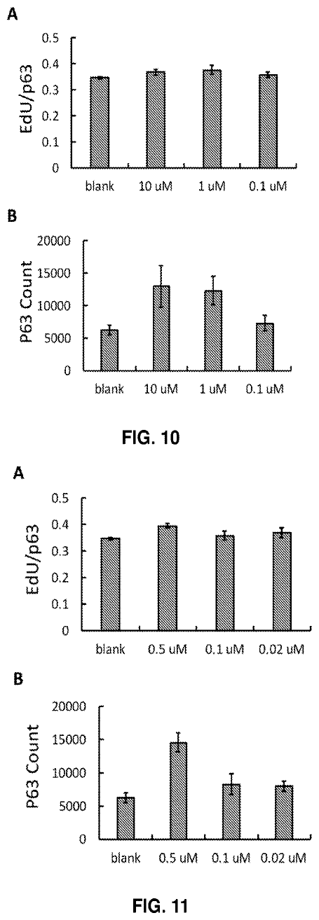

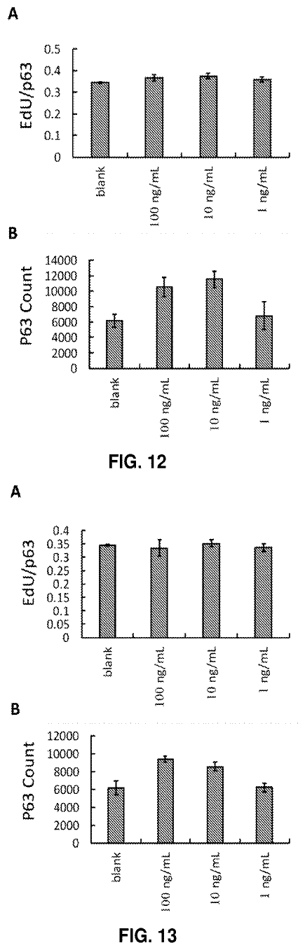

The result shows that at least 1 μM (such as 1-10 μM) of Y-27632 could promote adult KSCs proliferation (FIG. 10, panel A) with a two-fold change of the total cell number compared with the blank (FIG. 10, panel B). For the A 83-01, at least 0.5 μM could promote adult KSCs proliferation (FIG. 11). For EGF, at least 10 ng/mL (such as 10-100 ng/mL) could promote adult KSCs proliferation and result in the most cells (FIG. 12). For KGF, at least 10 ng/mL (such as 10-100 ng/mL) could increase the cell number (FIG. 13).

### Example 8—the Complete Medium

Here, 10 μM of Y-27632, 0.5 μM of A 83-01, 10 ng/mL of EGF, and 10 ng/mL of KGF were added as a supplement to the KSFX-free basal medium to make a KSFX-free complete medium.

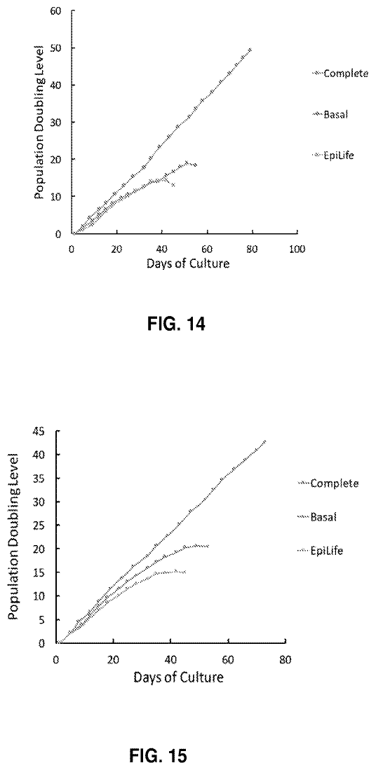

The long-term expansion of adult KSCs in the KSFX-free basal medium, the complete medium, as well as the Epilife with supplement S7, were compared. The result shows adult KSCs cultured in the complete medium grew faster and could proliferate to about 50 population doublings after 20 passages in vitro (FIG. 14). Adult KSCs cultured in the basal medium could proliferate to about 20 population doublings, while cells in EpiLife could only proliferate to less than 15 population doublings. The same result with the adult KSCs from a second donor is shown in FIG. 15.

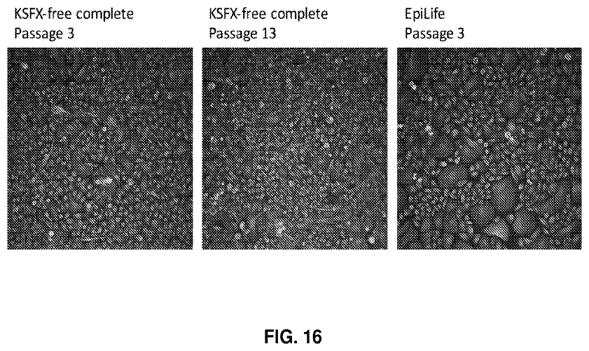

Keratin 14 (KRT14) is a prototypic marker of KSCs. It helps in the maintenance of epidermal cell shape, provides resistance to mechanical stress and inhibits KSCs from differentiation. To confirm the constant expansion of adult KSCs cultured in KSFX-free complete medium, the expression of KRT 14 in adult KSCs was measured. The result shows a similar expression level of KRT14 in adult KSCs at passage 3 and passage13 cultured in KSFX-free complete medium, as well as in EpiLife at passage 3 (FIG. 16). Furthermore, there were more small cells, which were thought to be undifferentiated and highly proliferative, present in KSFX-free complete medium than EpiLife. (FIG. 16).

Fetal keratinocytes may have faster expansion times, longer telomeres, lower immunogenicity indicators, and greater clonogenicity with more stem cell indicators than adult keratinocytes, suggesting potential allogeneic applications.

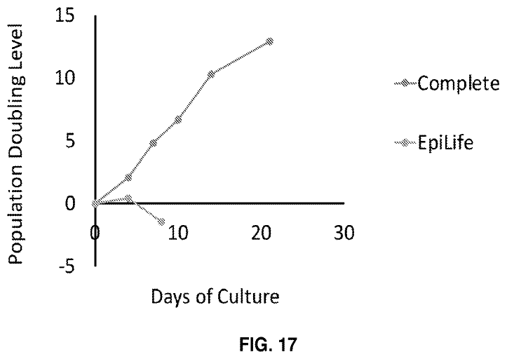

The long-term expansion of fetal KSCs in the KSFX-free complete medium and the Epilife with supplement S7 were compared. The result shows fetal KSCs cultured in the complete medium grew faster and could proliferate to about 14 population doublings, while cells in EpiLife could not grow or proliferate (FIG. 17).

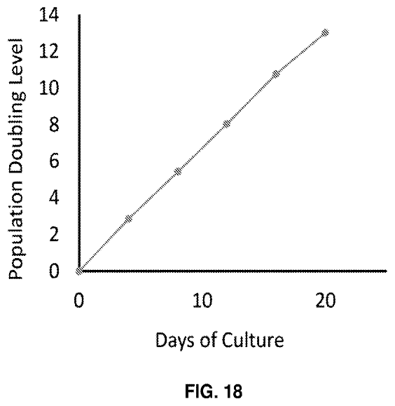

The isolation and in vitro culture of corneal epithelial stem cells were similar to those of KSCs described in Example 2 above. The long-term expansion of corneal epithelial stem cells in the KSFX-free complete medium was tested. The result shows corneal epithelial stem cells cultured in the complete medium could proliferate to about 14 population doublings (FIG. 18).

### Example 9—Coating Matrix Function Assay in the KSFX-Free Complete Medium

The function of coating matrix in the KSFX-free complete medium is tested. The tested coating matrixes include collagen, elastin, fibronectin, laminin, vitronectin, gelatin, poly-D-lysine, poly-L-ornithine, or any combination thereof (e.g., Matrigel).

The attachment and spreading of adult KSCs, fetal KSCs, and corneal epithelial stem cells in the KSFX-free complete medium on cell culture plate treated with each coating matrix are tested. The KSFX-free complete medium comprises 0.15 mM calcium, 10 μM of Y-27632, 0.5 μM of A 83-01, 10 ng/mL of EGF, and 10 ng/mL of KGF. Bright-field microscopy is performed at different time points (e.g., after 30 min, 2 hours, 6 hours, and 24 hours of culturing) to evaluate the attachment and spreading.

### Example 10—Calcium Concentration Function Assay in the KSFX-Free Complete Medium

The effect of calcium concentrations in the KSFX-free complete medium on the proliferation and differentiation of adult KSCs, fetal KSCs, and corneal epithelial stem cells are tested. Cells are cultured in different concentrations of calcium, including 0 mM, 0.05 mM, 0.15 mM, 0.5 mM, and 1.5 mM for 10 passages. The KSFX-free complete medium comprises 10 μM of Y-27632, 0.5 μM of A 83-01, 10 ng/mL of EGF, and 10 ng/mL of KGF. The proliferation and differentiation are then evaluated.

### Example 11—Combination of Growth Supplements Function Assay

The effect of combination of different growth supplements are tested. In the KSFK-free complete medium with 0.15 mM calcium, the concentration of Y-27632 is O μM, 0.1 μM, 1 μM, or 10 μM; the concentration of A 83-01 is 0 μM, 0.02 μM, 0.1 μM, or 0.5 μM; the concentration of EGF is O ng/mL, 1 ng/mL, 10 ng/mL, or 100 ng/mL; and the concentration of KGF is O ng/mL, 1 ng/mL, 10 ng/mL, or 100 ng/mL. Each of adult KSCs, fetal KSCs, and corneal epithelial stem cells are cultured in each one of the 256 culture media with different combinations of growth supplements. The proliferation and differentiation are then evaluated.

### Numbered Embodiments of the Disclosure

Other subject matter contemplated by the present disclosure is set out in the following numbered non-limiting embodiments:
1. A serum-free composition, comprising: a coating matrix, a basal medium, and a growth supplement.
2. The serum-free composition of embodiment 1, wherein the composition is substantially free of human or non-human animal serum.
3. The serum-free composition of embodiment 1 or 2, wherein the composition is animal component-free.
4. The serum-free composition of embodiment 1 or 2, wherein the composition is substantially free of mouse feeder layer, fetal bovine serum (FBS), bovine pituitary extract, or bovine serum albumin (BSA).
5. The serum-free composition of any one of embodiments 1-4, wherein the coating matrix comprises at least one extracellular matrix protein or synthetic polymer.
6. The serum-free composition of embodiment 5, wherein the at least one extracellular matrix protein comprises collagen, elastin, fibronectin, laminin, vitronectin, gelatin, or any combination thereof.
7. The serum-free composition of embodiment 6, wherein the collagen comprises collagen type I, II, or IV.
8. The serum-free composition of embodiment 6, wherein the fibronectin comprises a recombinant human fibronectin (rh-fibronectin).
9. The serum-free composition of embodiment 6, wherein the laminin comprises a recombinant human laminin (rh-laminin).
10. The serum-free composition of embodiment 5, wherein the at least one synthetic polymer comprises poly-D-lysine, poly-L-ornithine or both.
11. The serum-free composition of any one of embodiments 1-10, wherein the basal medium comprises glucose, amino acid, vitamin, sodium pyruvate, or any combination thereof.
12. The serum-free composition of any one of embodiments 1-11, wherein the basal medium further comprises L-glutamine, L-leucine, L-lysine, magnesium chloride, magnesium sulfate, L-methionine, sodium bicarbonate, calcium chloride, hydrocortisone, isoprenaline chloride, liothyronine, adenine, L-Alanyl-L-Glutamine, insulin, transferrin, sodium selenite, or any combination thereof.
13. The serum-free composition of any one of embodiments 1-12, wherein the composition has a final calcium concentration of at least about 0.1 mM.
14. The serum-free composition of embodiment 13, wherein the composition has a final calcium concentration of about 0.1-1.0 mM.
15. The serum-free composition of embodiment 14, wherein the composition has a final calcium concentration of about 0.15 mM.
16. The serum-free composition of any one of embodiments 1-15, wherein the growth supplement comprises at least one growth factor.
17. The serum-free composition of embodiment 16, wherein the at least one growth factor comprises epidermal growth factor (EGF), keratinocyte growth factor (KGF), or both.
18. The serum-free composition of embodiment 17, wherein the EGF comprises recombinant human epidermal growth factor (rh-EGF).
19. The serum-free composition of embodiment 17 or 18, wherein the composition has a final EGF concentration of about 0.1-300 ng/mL.
20. The serum-free composition of embodiment 19, wherein the composition has a final EGF concentration of about 10 ng/mL.
21. The serum-free composition of any one of embodiments 17-20, wherein the KGF comprises recombinant human keratinocyte growth factor (rh-KGF).
22. The serum-free composition of embodiment 21, wherein the composition has a final KGF concentration of about 0.1-300 ng/mL.
23. The serum-free composition of embodiment 22, wherein the composition has a final KGF concentration of about 10 ng/mL.
24. The serum-free composition of any one of embodiments 1-23, wherein the growth supplement further comprises a rho kinase (ROCK) inhibitor, a transforming growth factor-beta (TGFB) receptor inhibitor, or both.
25. The serum-free composition of embodiment 24, wherein the ROCK inhibitor comprises AT-13148, BA-210, β-elemene, DJ4, fasudil, GSK-576371, GSK429286A, H-1152, hydroxyfasudil, ibuprofen, LX-7101, netarsudil, RKI-1447, ripasudil, TCS-7001, thiazovivin, verosudil, Y-27632, Y-30141, Y-33075, Y-39983, or any combination thereof.
26. The serum-free composition of embodiment 25, wherein the composition has a final ROCK inhibitor concentration of about 0.1-100 μM.
27. The serum-free composition of embodiment 26, wherein the composition has a final ROCK inhibitor concentration of about 10 μM.
28. The serum-free composition of embodiment 24, wherein the TGFB receptor inhibitor comprises RepSox, SB-431542, A83-01, Galunisertib, or any combination thereof.
29. The serum-free composition of embodiment 28, wherein the composition has a final TGFB receptor inhibitor concentration of about 0.01-10 μM.
30. The serum-free composition of embodiment 29, wherein the composition has a final TGFB receptor inhibitor concentration of about 0.5 μM.
31. A method of preparing the serum-free composition in any one of embodiments 1-30, comprising: a) mixing the basal medium and growth supplement to form a mixture solution; and b) contacting the coating matrix with the mixture solution.
32. A method of culturing a plurality of cells in the serum-free composition in any one of embodiments 1-30, comprising: contacting the serum-free composition with the plurality of cells.
33. The method of embodiment 32, wherein the plurality of cells comprises stem cells.
34. The method of embodiment 33, wherein the plurality of cells comprises keratinocyte stem cells (KSCs).
35. The method of embodiment 34, wherein the KSCs comprise adult KSCs.
36. The method of embodiment 34, wherein the KSCs comprise fetal KSCs.
37. The method of embodiment 33, wherein the plurality of cells comprises corneal epithelial stem cells.
38. A kit, comprising a coating matrix, a basal medium, and a concentrated stock of a growth supplement.
39. The kit of embodiment 38, wherein the coating matrix is substantially free of mouse feeder layer.
40. The kit of embodiment 38 or 39, wherein the basal medium is substantially free of human or non-human animal serum.
41. The kit of any one of embodiments 38-40, wherein the coating matrix, basal medium, and growth supplement are substantially free of cells or components derived from non-human animals.
42. The kit of any one of embodiments 38-40, wherein the coating matrix, basal medium, and growth supplement are substantially free of mouse feeder layer, fetal bovine serum (FBS), bovine pituitary extract, or bovine serum albumin (BSA).
43. The kit of any one of embodiments 38-42, wherein the coating matrix comprises at least one extracellular matrix protein or synthetic polymer.
44. The kit of embodiment 43, wherein the at least one extracellular matrix protein comprises collagen, elastin, fibronectin, laminin, vitronectin, gelatin, or any combination thereof.
45. The kit of embodiment 44, wherein the collagen comprises collagen type I, II, or IV.
46. The kit of embodiment 44, wherein the fibronectin comprises a recombinant human fibronectin (rh-fibronectin).
47. The kit of embodiment 44, wherein the laminin comprises a recombinant human laminin (rh-laminin).
48. The kit of embodiment 43, wherein the at least one synthetic polymer comprises poly-D-lysine, poly-L-ornithine or both.
49. The kit of any one of embodiments 38-48, wherein the basal medium comprises glucose, amino acid, vitamin, sodium pyruvate, or any combination thereof.
50. The kit of embodiment 49, wherein the basal medium further comprises L-glutamine, L-leucine, L-lysine, magnesium chloride, magnesium sulfate, L-methionine, sodium bicarbonate, calcium chloride, hydrocortisone, isoprenaline chloride, liothyronine, adenine, L-Alanyl-L-Glutamine, insulin, transferrin, sodium selenite, or any combination thereof.
51. The kit of any one of embodiments 38-50, wherein the basal medium has a final calcium concentration of at least about 0.1 mM.
52. The kit of embodiment 51, wherein the basal medium has a final calcium concentration of about 0.1-1.0 mM.
53. The kit of embodiment 52, wherein the basal medium has a final calcium concentration of about 0.15 mM.
54. The kit of any one of embodiments 38-53, wherein the growth supplement comprises at least one growth factor.
55. The kit of embodiment 54, wherein the at least one growth factor comprises epidermal growth factor (EGF), keratinocyte growth factor (KGF), or both.
56. The kit of embodiment 55, wherein the EGF comprises recombinant human epidermal growth factor (rh-EGF).
57. The kit of embodiment 55 or 56, wherein the concentrated stock of the growth supplement has a final EGF concentration of about 0.1-50 μg/mL.
58. The kit of embodiment 57, wherein the concentrated stock of the growth supplement has a final EGF concentration of about 10 μg/mL.
59. The kit of embodiment 55, wherein the KGF comprises recombinant human keratinocyte growth factor (rh-KGF).
60. The kit of embodiment 59, wherein the concentrated stock of the growth supplement has a final KGF concentration of about 0.1-50 μg/mL.
61. The kit of embodiment 60, wherein the concentrated stock of the growth supplement has a final KGF concentration of about 10 μg/mL.
62. The kit of any one of embodiments 38-61, wherein the growth supplement further comprises a rho kinase (ROCK) inhibitor, a transforming growth factor-beta (TGFB) receptor inhibitor, or both.
63. The kit of embodiment 62, wherein the ROCK inhibitor comprises AT-13148, BA-210, β-elemene, DJ4, fasudil, GSK-576371, GSK429286A, H-1152, hydroxyfasudil, ibuprofen, LX-7101, netarsudil, RKI-1447, ripasudil, TCS-7001, thiazovivin, verosudil, Y-27632, Y-30141, Y-33075, Y-39983, or any combination thereof.
64. The kit of embodiment 63, wherein the concentrated stock of the growth supplement has a final ROCK inhibitor concentration of about 0.1-50 mM.
65. The kit of embodiment 64, wherein the concentrated stock of the growth supplement has a final ROCK inhibitor concentration of about 10 mM.
66. The kit of embodiment 62, wherein the TGFB receptor inhibitor comprises RepSox, SB-431542, A83-01, Galunisertib, or any combination thereof.
67. The kit of embodiment 66, wherein the composition has a final TGFB receptor inhibitor concentration of about 0.1-5.0 mM.
68. The kit of embodiment 67, wherein the composition has a final TGFB receptor inhibitor concentration of about 0.5 mM.

The details of one or more inventive embodiments are set forth in the accompanying drawings, the claims, and the description herein. Other features, objects, and advantages of the inventive embodiments disclosed and contemplated herein can be combined with any other embodiment unless explicitly excluded. The various embodiments described above can be combined to provide further embodiments. All of the U.S. patents, U.S. patent application publications, U.S. patent application, foreign patents, foreign patent application and non-patent publications referred to in this specification and/or listed in the Application Data Sheet are incorporated herein by reference, in their entirety.

What is claimed is:

1. A serum-free composition, comprising: a coating matrix, a basal medium, and a growth supplement.

2. The serum-free composition of claim 1, wherein the composition is substantially free of human or non-human animal serum.

3. The serum-free composition of claim 1 or 2, wherein the composition is animal component-free.

4. The serum-free composition of claim 1 or 2, wherein the composition is substantially free of mouse feeder layer, fetal bovine serum (FBS), bovine pituitary extract, or bovine serum albumin (BSA).

5. The serum-free composition of any one of claims 1-4, wherein the coating matrix comprises at least one extracellular matrix protein or synthetic polymer.

6. The serum-free composition of claim 5, wherein the at least one extracellular matrix protein comprises collagen, elastin, fibronectin, laminin, vitronectin, gelatin, or any combination thereof.

7. The serum-free composition of claim 6, wherein the collagen comprises collagen type I, II, or IV.

8. The serum-free composition of claim 6, wherein the fibronectin comprises a recombinant human fibronectin (rh-fibronectin).

9. The serum-free composition of claim 6, wherein the laminin comprises a recombinant human laminin (rh-laminin).

10. The serum-free composition of claim 5, wherein the at least one synthetic polymer comprises poly-D-lysine, poly-L-ornithine or both.

11. The serum-free composition of any one of claims 1-10, wherein the basal medium comprises glucose, amino acid, vitamin, sodium pyruvate, or any combination thereof.

12. The serum-free composition of any one of claims 1-11, wherein the basal medium further comprises at least one of L-glutamine, L-leucine, L-lysine, magnesium chloride, magnesium sulfate, L-methionine, sodium bicarbonate, calcium chloride, hydrocortisone, isoprenaline chloride, liothyronine, adenine, L-Alanyl-L-Glutamine, insulin, transferrin, sodium selenite.

13. The serum-free composition of any one of claims 1-12, wherein the composition has a final calcium concentration of at least about 0.1 mM.

14. The serum-free composition of claim 13, wherein the composition has a final calcium concentration of about 0.1-1.0 mM.

15. The serum-free composition of claim 14, wherein the composition has a final calcium concentration of about 0.15 mM.

16. The serum-free composition of any one of claims 1-15, wherein the growth supplement comprises at least one growth factor.

17. The serum-free composition of claim 16, wherein the at least one growth factor comprises epidermal growth factor (EGF), keratinocyte growth factor (KGF), or both.

18. The serum-free composition of claim 17, wherein the EGF comprises recombinant human epidermal growth factor (rh-EGF).

19. The serum-free composition of claim 17 or 18, wherein the composition has a final EGF concentration of about 0.1-300 ng/mL.

20. The serum-free composition of claim 19, wherein the composition has a final EGF concentration of about 10 ng/mL.

21. The serum-free composition of any one of claims 17-20, wherein the KGF comprises recombinant human keratinocyte growth factor (rh-KGF).

22. The serum-free composition of claim 21, wherein the composition has a final KGF concentration of about 0.1-300 ng/mL.

23. The serum-free composition of claim 22, wherein the composition has a final KGF concentration of about 10 ng/mL.

24. The serum-free composition of any one of claims 1-23, wherein the growth supplement further comprises a rho kinase (ROCK) inhibitor, a transforming growth factor-beta (TGFB) receptor inhibitor, or both.

25. The serum-free composition of claim 24, wherein the ROCK inhibitor comprises at least one of AT-13148, BA-210, β-elemene, DJ4, fasudil, GSK-576371, GSK429286A, H-1152, hydroxyfasudil, ibuprofen, LX-7101, netarsudil, RKI-1447, ripasudil, TCS-7001, thiazovivin, verosudil, Y-27632, Y-30141, Y-33075, Y-39983.

26. The serum-free composition of claim 25, wherein the composition has a final ROCK inhibitor concentration of about 0.1-100 μM.

27. The serum-free composition of claim 26, wherein the composition has a final ROCK inhibitor concentration of about 10 μM.

28. The serum-free composition of claim 24, wherein the TGFB receptor inhibitor comprises at least one of RepSox, SB-431542, A83-01, Galunisertib.

29. The serum-free composition of claim 28, wherein the composition has a final TGFB receptor inhibitor concentration of about 0.01-10 μM.

30. The serum-free composition of claim 29, wherein the composition has a final TGFB receptor inhibitor concentration of about 0.5 μM.

31. A method of preparing the serum-free composition in any one of claims 1-30, comprising: a) mixing the basal medium and growth supplement to form a mixture solution; and b) contacting the coating matrix with the mixture solution.

32. A method of culturing a plurality of cells in the serum-free composition in any one of claims 1-30, comprising: contacting the serum-free composition with the plurality of cells.

33. The method of claim 32, wherein the plurality of cells comprises stem cells.

34. The method of claim 33, wherein the plurality of cells comprises keratinocyte stem cells (KSCs).

35. The method of claim 34, wherein the KSCs comprise adult KSCs.

36. The method of claim 34, wherein the KSCs comprise fetal KSCs.

37. The method of claim 33, wherein the plurality of cells comprises corneal epithelial stem cells.

38. A kit, comprising a coating matrix, a basal medium, and a concentrated stock of a growth supplement.

39. The kit of claim 38, wherein the coating matrix is substantially free of mouse feeder layer.

40. The kit of claim 38 or 39, wherein the basal medium is substantially free of human or non-human animal serum.

41. The kit of any one of claims 38-40, wherein the coating matrix, basal medium, and growth supplement are substantially free of cells or components derived from non-human animals.

42. The kit of any one of claims 38-40, wherein the coating matrix, basal medium, and growth supplement are substantially free of mouse feeder layer, fetal bovine serum (FBS), bovine pituitary extract, or bovine serum albumin (BSA).

43. The kit of any one of claims 38-42, wherein the coating matrix comprises at least one extracellular matrix protein or synthetic polymer.

44. The kit of claim 43, wherein the at least one extracellular matrix protein comprises at least one of collagen, elastin, fibronectin, laminin, vitronectin, gelatin.

45. The kit of claim 44, wherein the collagen comprises collagen type I, II, or IV.

46. The kit of claim 44, wherein the fibronectin comprises a recombinant human fibronectin (rh-fibronectin).

47. The kit of claim 44, wherein the laminin comprises a recombinant human laminin (rh-laminin).

48. The kit of claim 43, wherein the at least one synthetic polymer comprises poly-D-lysine, poly-L-ornithine or both.

49. The kit of any one of claims 38-48, wherein the basal medium comprises at least one of glucose, amino acid, vitamin, sodium pyruvate.

50. The kit of claim 49, wherein the basal medium further comprises at least one of L-glutamine, L-leucine, L-lysine, magnesium chloride, magnesium sulfate, L-methionine, sodium bicarbonate, calcium chloride, hydrocortisone, isoprenaline chloride, liothyronine, adenine, L-Alanyl-L-Glutamine, insulin, transferrin, sodium selenite.

51. The kit of any one of claims 38-50, wherein the basal medium has a final calcium concentration of at least about 0.1 mM.

52. The kit of claim 51, wherein the basal medium has a final calcium concentration of about 0.1-1.0 mM.

53. The kit of claim 52, wherein the basal medium has a final calcium concentration of about 0.15 mM.

54. The kit of any one of claims 38-53, wherein the growth supplement comprises at least one growth factor.

55. The kit of claim 54, wherein the at least one growth factor comprises epidermal growth factor (EGF), keratinocyte growth factor (KGF), or both.

56. The kit of claim 55, wherein the EGF comprises recombinant human epidermal growth factor (rh-EGF).

57. The kit of claim 55 or 56, wherein the concentrated stock of the growth supplement has a final EGF concentration of about 0.1-50 μg/mL.

58. The kit of claim 57, wherein the concentrated stock of the growth supplement has a final EGF concentration of about 10 μg/mL.

59. The kit of claim 55, wherein the KGF comprises recombinant human keratinocyte growth factor (rh-KGF).

60. The kit of claim 59, wherein the concentrated stock of the growth supplement has a final KGF concentration of about 0.1-50 μg/mL.

61. The kit of claim 60, wherein the concentrated stock of the growth supplement has a final KGF concentration of about 10 μg/mL.

62. The kit of any one of claims 38-61, wherein the growth supplement further comprises a rho kinase (ROCK) inhibitor, a transforming growth factor-beta (TGFB) receptor inhibitor, or both.

63. The kit of claim 62, wherein the ROCK inhibitor comprises AT-13148, BA-210, 3-elemene, DJ4, fasudil, GSK-576371, GSK429286A, H-1152, hydroxyfasudil, ibuprofen, LX-7101, netarsudil, RKI-1447, ripasudil, TCS-7001, thiazovivin, verosudil, Y-27632, Y-30141, Y-33075, Y-39983, or any combination thereof.

64. The kit of claim 63, wherein the concentrated stock of the growth supplement has a final ROCK inhibitor concentration of about 0.1-50 mM.

65. The kit of claim 64, wherein the concentrated stock of the growth supplement has a final ROCK inhibitor concentration of about 10 mM.

66. The kit of claim 62, wherein the TGFB receptor inhibitor comprises RepSox, SB-431542, A83-01, Galunisertib, or any combination thereof.

67. The kit of claim 66, wherein the composition has a final TGFB receptor inhibitor concentration of about 0.1-5.0 mM.

68. The kit of claim 67, wherein the composition has a final TGFB receptor inhibitor concentration of about 0.5 mM.
